(19) United States
(12) Patent Application Publication
(10) Pub. No.: US 2016/0059006 A1
(43) Pub. Date: Mar. 3, 2016

(54) CARDIAC LEAD WITH SNAP-LOCK CONSTRUCTION OF INTEGRATED DISTAL TIP ASSEMBLY

(71) Applicant: PACESETTER, INC., Sylmar, CA (US)

(72) Inventors: Phong D. Doan, San Clemente, CA (US); Virote Indravudh, Santa Clarita, CA (US); Steven R. Conger, Agua Dulce, CA (US); Michael Childers, Montrose, CA (US); Daniel Hale, Studio City, CA (US)

(21) Appl. No.: 14/469,359

(22) Filed: Aug. 26, 2014

(51) Int. Cl. A61N 1/05

(52) U.S. Cl. CPC A61N 1/0565; A61N 1/0573

(57) ABSTRACT

An implantable therapy lead is disclosed herein. The therapy lead includes an integrated distal tip assembly with a header. At least one of a helix-shaft assembly, a ring electrode or a marker ring is directly coupled to the header via a snap-lock coupling arrangement.

### FIELD OF THE INVENTION

The present invention relates to medical apparatus and methods. More specifically, the present invention relates to implantable therapy leads and methods of assembling such leads.

### BACKGROUND OF THE INVENTION

Implantable therapy leads may be configured for active fixation. A common arrangement for a lead configured for active fixation provides a lead distal end with an active fixation helix that extends from the distal end of the lead when a contact pin is rotated at a proximal end of the lead. As the contact pin is rotated about its longitudinal axis, the sharp helix rotates and extends from the lead distal end to screw into myocardial tissue. In some other embodiments, a stylet or other tool is inserted through the lead body to deploy the active fixation helix via rotation and/or sliding distal displacement of the active fixation helix brought about by complementary interaction of the stylet or other tool with structural features of, or associated with, the active fixation helix.

In addition to serving as a mechanism that anchors the lead distal end to myocardial tissue, the helix can also serve as an electrode for pacing and sensing functions of the lead.

Such active fixation helix arrangements are mechanically complex and expensive to manufacture. Accordingly, there is a need in the art for an active fixation lead that is more cost effective to manufacture.

### SUMMARY

An implantable therapy lead is disclosed herein. In one embodiment, the therapy lead includes an integrated distal tip assembly with a header. At least one of a helix-shaft assembly, a ring electrode or a marker ring is directly coupled to the header via a snap-lock coupling arrangement.

In one version of the lead embodiment, the helix-shaft assembly, in addition to being directly coupled to the header via the snap-lock coupling arrangement, is also longitudinally displaceable relative to the header. The snap-lock coupling arrangement between the header and the helix-shaft assembly may include a tapered male member on the helix-shaft assembly and a female throat in the header. The female throat will have received, and snap-locked with, the tapered male member during an assembly process.

In one version of the lead embodiment, the helix-shaft assembly includes a flexible O-ring supported on the helix-shaft assembly adjacent the tapered male member and in a sliding sealed engagement with the header proximal the female throat.

In one version of the lead embodiment, the female throat includes a cantilevered structure that was forced radially outward by the passage of the tapered male member through the female throat during the assembly process. The cantilevered structure will have biased radially inward to snap-lock with the tapered male member after the tapered male member cleared the cantilevered structure. The cantilevered structure may distally project, and the tapered male member may taper in a proximal direction.

In one version of the lead embodiment, the snap-lock coupling arrangement between the header and the ring electrode includes: a male end of the header received in a female end of the ring electrode; and a radially inwardly biased tab defined in the ring electrode that is received in, and snap-locked with, an opening or recess defined in the male end of the header. The radially inwardly biased tab may have a cantilevered configuration that includes a proximally facing free end that is received in, and snap-locks with, the opening or recess defined in the male end of the header.

In one version of the lead embodiment, the snap-lock coupling arrangement between the header and the ring electrode includes: a male end of the header received in a female end of the ring electrode; and a male tab defined in the male end of the header that is received in, and snap-locked with, an opening or recess defined in the ring electrode. The male tab may include a sloped proximal surface.

In one version of the lead embodiment, the snap-lock coupling arrangement between the header and the marker ring includes: a male end of the header received in a female end of the marker ring; and a radially inwardly biased tab defined in the marker ring that is received in, and snap-locked with, an opening or recess defined in the male end of the header. The radially inwardly biased tab may have a cantilevered configuration that includes a distally facing free end that is received in, and snap-locks with, the opening or recess defined in the male end of the header.

In one version of the lead embodiment, the snap-lock coupling arrangement between the header and the marker ring includes: a male end of the header received in a female end of the marker ring; and a male tab defined in the male end of the header that is received in, and snap-locked with, an opening or recess defined in the marker ring. The male tab may include a sloped distal surface. An atraumatic tip may be overmolded on the marker ring.

In one version of the lead embodiment, the snap-lock coupling arrangement between the header and the ring electrode includes: a male end of the header received in a female end of the ring electrode; and a radially inwardly biased tab defined in the ring electrode that is received in, and snap-locked with, an opening or recess defined in the male end of the header, the opening intersecting a thread feature defined in the male end of the header.

In one version of the lead embodiment, the snap-lock coupling arrangement between the header and the marker ring includes: a male end of the header received in a female end of the marker ring; and a radially inwardly biased tab defined in the marker ring that is received in, and snap-locked with, an opening or recess defined in the male end of the header, the opening intersecting a thread feature defined in the male end of the header.

In one version of the lead embodiment, the snap-lock coupling arrangement between the header and the marker ring includes: a male end of the header received in a female end of the ring electrode; and a first longitudinally extending structural feature on the marker ring that mates with, and slides along, a second longitudinally extending structural feature of the male end of the header. The first longitudinally extending structural feature may include a slot, and the second longitudinally extending structural feature may include a raised ridge.

In one version of the lead embodiment, the snap-lock coupling arrangement between the header and the ring electrode includes: a male end of the header received in a female end of the ring electrode; and a first longitudinally extending structural feature on the ring electrode that mates with, and slides along, a second longitudinally extending structural feature of the male end of the header. The first longitudinally extending structural feature may include a slot, and the second longitudinally extending structural feature may include a raised ridge.

A method of assembling an integrated distal tip assembly of an implantable therapy lead is also disclosed herein. In one embodiment, the method includes directly coupling at least one of a helix-shaft assembly, a ring electrode or a marker ring to a header via a snap-lock coupling arrangement.

In one version of the method embodiment, a helical conductor proximally extends from a proximal region of the helix-shaft assembly. The helix-shaft assembly is directly coupled to the header by extending a proximal end of the helical conductor through a throat of the header followed by proximally extending the helix-shaft assembly through the throat until a tapered male member of the helix-shaft passes through the throat and engages with the throat in the snap-lock coupling arrangement.

In one version of the method embodiment, the method further includes inserting a distal male end of the header into a proximal female end of a marker ring. The snap-lock coupling arrangement between the header and the marker ring occurs as at least one of: a male tab of the marker ring is received in a recess or opening of the header; or a male tab of the header is received in a recess or opening of the marker ring.

In one version of the method embodiment, the method further includes inserting a proximal male end of the header into a distal female end of a ring electrode. The snap-lock coupling arrangement between the header and the ring electrode occurs as at least one of: a male tab of the ring electrode is received in a recess or opening of the header; or a male tab of the header is received in a recess or opening of the ring electrode.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a plan view of an embodiment of a lead, wherein an active fixation anchor of the lead is shown in an extended or deployed state.

FIG. 2A is a longitudinal cross-section of a distal region of the lead of FIG. 1, wherein the active fixation anchor is shown in the extended or deployed state.

FIG. 2B is the same view as FIG. 2A, except the active fixation anchor is shown in a retracted or non-deployed state.

FIG. 3 is an exploded isometric longitudinal cross-section of a header and helix-shaft assembly of an integrated distal tip assembly of the distal region of FIG. 2A.

FIG. 4 is an enlarged view of a throat structure region illustrated in FIG. 3.

FIG. 5 is an enlarged view of a throat engagement structure illustrated in FIG. 3.

FIG. 6A is an exploded isometric view of the of the ring electrode, header and the marker tip assembly, wherein the header has female snap-lock features and the ring electrode and the marker tip assembly have complementary male snap-lock features.

FIG. 6B is a view similar to that of FIG. 6A, except of another embodiment wherein the header has male snap-lock features and the ring electrode and the marker tip assembly have complementary female snap-lock features.

FIGS. 7A and 7B are, respectively, isometric and longitudinal cross sectional views of the marker tip assembly.

FIG. 7C is a longitudinal cross section of the marker ring illustrating a female snap-lock feature in the form of a cut out.

FIGS. 8A and 8B are, respectively, side elevation and longitudinal cross sectional views of the distal region of the header distally terminating in the header distal end.

FIGS. 9A and 9B are, respectively, isometric and longitudinal cross sectional views of the ring electrode.

FIG. 9C is a longitudinal cross section of the ring electrode having a press-fit weld collar.

FIGS. 10A and 10B are, respectively, isometric and longitudinal cross sectional views of the proximal region of the header proximally terminating in the header proximal end.

FIG. 10C is a side view of the proximal region of the header proximally terminating in the header proximal end, except the snap-lock features thereon are male snap-lock features.

FIG. 11 is the same view as FIG. 2A, except of an alternative embodiment wherein the lead distal tip assembly employs a post-less design.

FIG. 12 is a cross section of a snap-lock arrangement,

FIG. 13 is an isometric view of another embodiment of the header proximal end, wherein the header proximal end includes threading and a locking feature at the end of the threading.

FIG. 14 is an exploded isometric view of additional embodiments of the electrode ring and header proximal end.

FIG. 15A is a side elevation view of the distal region of the lead via fluoroscopic visualization, wherein the helical anchor is shown in the non-deployed state.

FIG. 15B is the same view as FIG. 15A, except the helical anchor is shown in the fully-deployed state.

### DETAILED DESCRIPTION

Implantable therapy leads 10 (e.g., a CRT lead, etc.) and methods of manufacturing such leads are disclosed herein. In one embodiment, the therapy lead 10 is configured for active fixation to heart tissue. The lead 10 includes a tubular body 12 and an integrated distal tip assembly 50. The integrated distal tip assembly 50 includes a ring electrode 30, a header 52, a helix-shaft assembly 54, and a marker ring assembly 55. The helix-shaft assembly 54 includes a shaft 58 and a helical active fixation anchor 26 extending distally therefrom. The marker ring assembly 55 includes a marker ring 56 and a soft atraumatic tip 57 extending distally therefrom.

The ring electrode 30, header 52, helix-shaft assembly 54, and marker ring assembly 55 are secured together via interference fit arrangements, some of which may be in the form of male-female interference fit arrangements including those employing snap-lock arrangements, for example. Such interference fit assembled integrated distal tip assemblies 50 as disclosed herein simplify and reduce the manufacturing costs as compared to those of traditional lead distal tip assemblies.

In other words, the integration of different parts into a single component and the addition of snap-lock features reduces manufacturing time and costs. Additionally, the use of low cost polyaryletherketone (“PEEK”) molded polymer and stamped-progressive formed parts in place of the traditional machined parts further reduces manufacturing costs.

Finally, in some embodiments, the distal tip assembly 50 disclosed herein includes a blood seal 172 and a helix-extension visibility configuration for visualization under fluoroscopy.

a. Overview of Lead

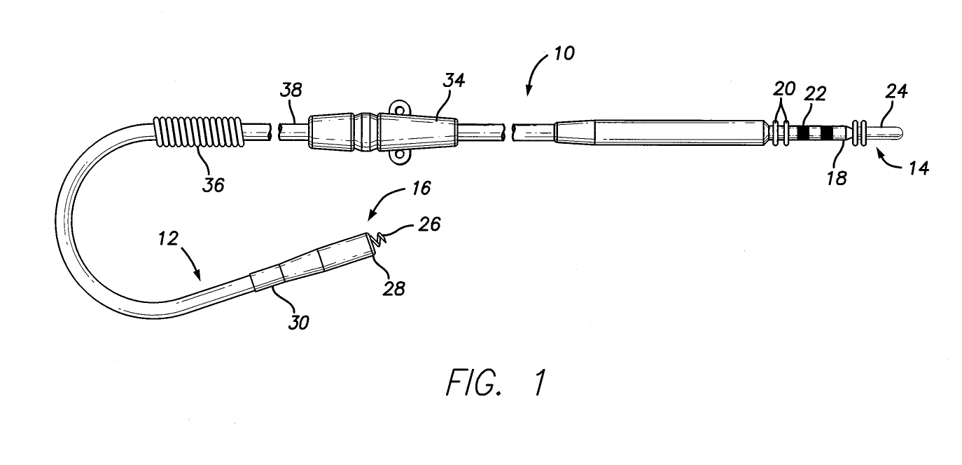

To begin a detailed discussion of the lead 10, reference is made to FIG. 1, which is a plan view of an embodiment of the lead 10 wherein an active fixation anchor 26 of the lead is shown in an extended or deployed state. As can be understood from FIG. 1, the lead 10 is designed for intravenous insertion and contact with the endocardium, and as such, may be conventionally referred to as an endocardial lead. As indicated in FIG. 1, the lead 10 is provided with an elongated lead body 12 that extends between a proximal region 14 and distal region 16 of the lead 10.

The proximal region 14 of the lead 10 includes a connector assembly 18, which is provided with sealing rings 20 and carries at least one or more electrical connectors in the form of ring contacts 22 and a pin contact 24. The connector assembly 18 is configured to be plugged into a receptacle of a pulse generator, the sealing rings 20 forming a fluid-tight seal to prevent the ingress of fluids into the receptacle of the pulse generator. When the connector assembly 18 is plugged into the pulse generator receptacle, the contacts 22, 24 electrically connect with the circuitry of the pulse generator such that electrical signals can be administered and sensed by the pulse generator via the electrical pathways of the lead 10.

The connector assembly 28 is constructed using known techniques and is preferably fabricated of silicone rubber, polyurethane, silicone-rubber-polyurethane-copolymer (“SPC”), or other suitable polymer. The electrical contacts 22, 24 are preferably fabricated of stainless steel or other suitable electrically conductive material that is biocompatible.

As shown in FIG. 1, the distal region 16 of the lead 20 includes the helical active fixation anchor 26 distally extending from an extreme distal tip end 28 of the lead 20 when the active fixation anchor 26 is in a deployed state. The anchor 26 may be transitioned to a non-deployed state via retraction of the anchor 26 into the confines of the distal region 16 of the lead 10 or by an obturator or other structural member being combined with the anchor 26 to inhibit the anchor 26 from being able to penetrate tissue.

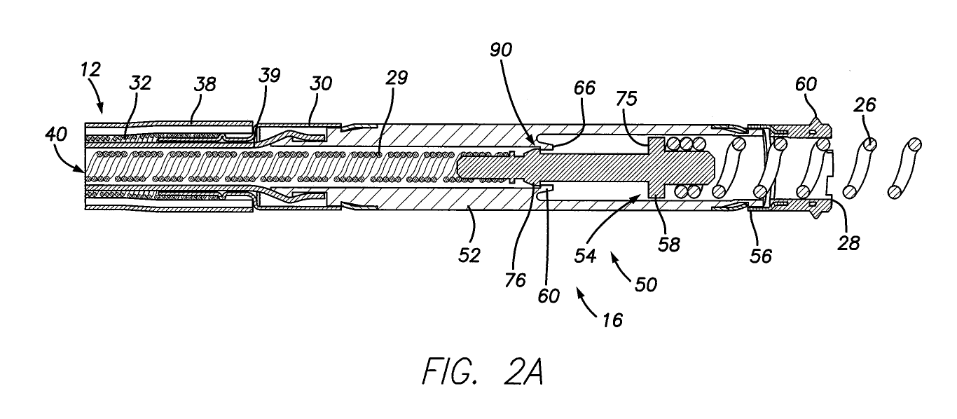

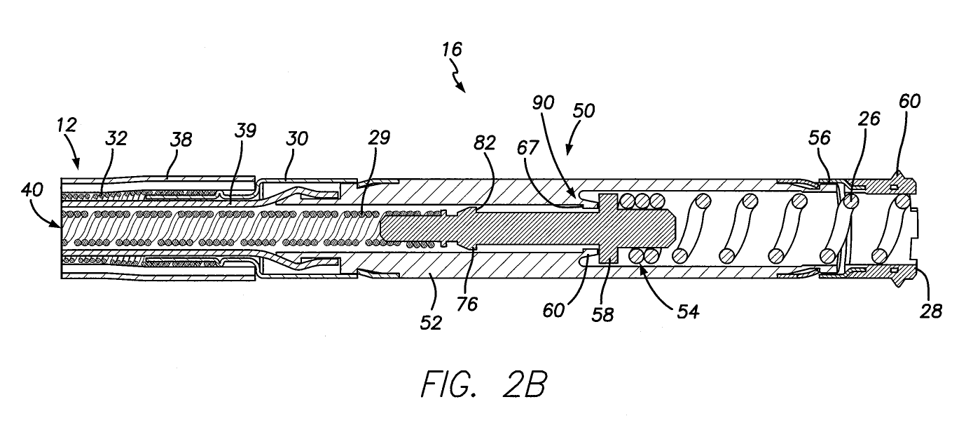

In one embodiment, the anchor 26 is deployed or placed in the extended state by rotating the contact pin 24, which is coupled via a helical conductor 29 (see FIGS. 2A and 2B) to the anchor 26. As the contact pin 24 is rotated about its longitudinal axis, the helical conductor 29 and sharp helical anchor 26 rotate relative to the rest of the lead 10 to cause the anchor 26 to extend from the lead distal end 28 to screw into myocardial tissue. In some other embodiments, a stylet or other tool is inserted through the lead body 12 to deploy the anchor 26 via rotation and/or sliding distal displacement of the anchor 26 brought about by complementary interaction of the stylet or other tool with structural features of, or associated with, the anchor 26.

The anchor 26 may also be configured to act as an electrode in addition to providing active fixation to heart tissue. Where the anchor 26 is also configured to act as an electrode, depending on the dictates of the pulse generator, the anchor 26 may be employed for sensing electrical energy and/or administration of electrical energy (e.g., pacing). The anchor 26 is electrically coupled to the pin contact 24 of the connector assembly 18 via the electrical conductor 29 extending through the lead body 12 and the connector assembly 18, as can be understood from FIGS. 2A and 2B, which are longitudinal cross-section of the distal region 16 of the lead 10 of FIG. 1, wherein the active fixation anchor 26 is shown in the extended or deployed state and the retracted or non-deployed state, respectively. While the electrical conductor 29 is shown as helically coiled electrical conductors, in other embodiments the conductor 29 may be in the form of wires, cables or other electrical conductors that are linear or helically coiled in configuration.

The distal region 16 of the lead 10 also includes an annular ring electrode 30 proximally offset from the extreme distal tip end 28 of the lead 10. Depending on the dictates of the pulse generator, this ring electrode 30 may be employed for sensing electrical energy and/or administration of electrical energy (e.g., pacing). The ring electrode 30 is electrically coupled to one of the ring contacts 22 of the connector assembly 18 via an electrical conductor 32 extending through the lead body 12 and the connector assembly 18, as can be understood from FIGS. 2A and 2B. While the electrical conductor 32 is shown as helically coiled electrical conductors, in other embodiments the conductor 32 may be in the form of wires, cables or other electrical conductors that are linear or helically coiled in configuration.

As indicated in FIG. 1, the lead 10 may include a fixation sleeve 34 slidably mounted around the lead body 12. The fixation sleeve 34 serves to stabilize the pacing lead 10 at the site of venous insertion.

Where the lead 10 is equipped for defibrillation, a shock coil 36 will be supported on the lead body 12 proximal the ring electrode 30 and distal the fixation sleeve 34. The shock coil 36 is electrically coupled to one of the ring contacts 22 of the connector assembly 18 via electrical conductors extending through the lead body 12 in the form of wires, cables or other electrical conductors that are linear or helically coiled in configuration.

As can be understood from FIGS. 1-2B, the lead body 12 includes an outer insulation sheath 38 and an inner insulation sheath 39. The outer insulation sheath 38 is preferably fabricated of silicone rubber, polyurethane, silicone rubber-polyurethane-copolymer (SPC), or other suitable polymer. The inner insulation sheath 39 may be formed of the same material as the outer insulation sheath 39 or from another material such as, for example, polytetrafluoroethylene (“PTFE”). The insulation sheaths 38, 39 isolate the interior components of the lead 10, including the electrical conductors 29, 32, from each other. The outer insulation sheath 38 isolates the inner components of the lead 10 from the surrounding environment and may be single or multi-layer construction.

The lead body 12 is constructed to include a hollow interior 40 extending from the proximal region 14 to the distal region 16. The hollow interior allows for the introduction of a stylet, guidewire or other device during implant, which is beneficial in allowing the surgeon to guide the otherwise flexible lead 10 from the point of venous insertion to the myocardium.

b. Integrated Distal Tip Assembly

As indicated in FIGS. 2A and 2B, the distal region 16 of the lead 10 includes an integrated distal tip assembly 50, which is the integration of different components into a single assembly 50 via interference fit or locking mechanical arrangements between the different components. The integrated distal tip assembly 50 is advantageous in that it has reduced associated manufacturing time and costs as compared to traditional distal tip assemblies known in the art.

The integrated distal tip assembly 50 includes a number of components, such as, for example, a ring electrode 30, a header 52, a helix-shaft assembly 54 and a marker ring assembly 55. The helix-shaft assembly 54 includes a shaft 58 and a helical active fixation anchor 26 extending distally therefrom. The marker ring assembly 55 includes a marker ring 56 and a soft atraumatic tip 57 extending distally therefrom. The ring electrode 30, header 52, helix-shaft assembly 54 and a marker ring assembly 55 are secured together via interference fit arrangements, some of which may be in the form of male-female interference fit arrangements including those employing snap-lock arrangements, for example.

1. Engagement of Header and Helix-Shaft Assembly

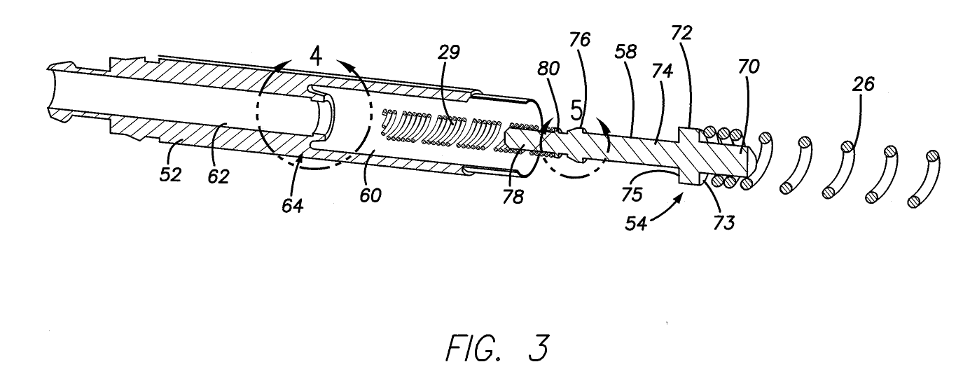

FIG. 3 is an exploded isometric longitudinal cross-section of the header 52 and helix-shaft assembly 54 of the integrated distal tip assembly 50. As can be understood from FIGS. 2A, 2B and 3, the helix-shaft assembly 54 includes a shaft 58 and the helical active fixation anchor 26 extending distally therefrom. The header 52 includes a distal cylindrical passage 60 and proximal cylindrical passage 62 that coaxially intersect with each other at a throat structure 64. The distal passage 60 has a larger diameter than the proximal passage 62.

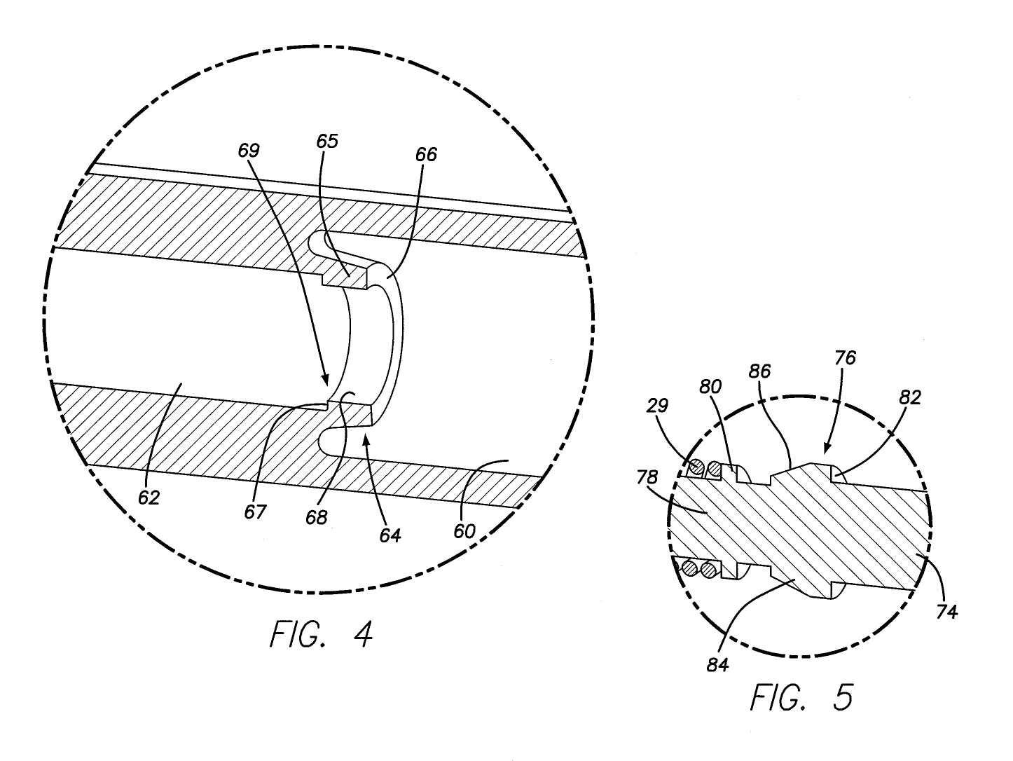

As shown in FIG. 4, which is an enlarged view of throat structure region illustrated in FIG. 3, the throat structure 64 includes a cantilevered ring-like structure 65 that distally projects into the volume of the distal cylindrical passage 60 from the distal boundary of the proximal cylindrical passage 62. The ring-like structure 65 includes a ringed blunt free end 66 that projects distally in the distal passage 60 as part of the cantilevered configuration of the throat structure 64. Opposite the free end 66, the ring-like structure 65 includes a proximally facing surface 67 that extends perpendicularly between the circumferential surface of the proximal passage 62 and a circumferential surface of a throat passage 68 that extends through the throat 64, thereby defining a ringed lip 69. The throat passage 68 is coaxial with the distal and proximal passages 60, 62, and is smaller in diameter than the proximal passage 62.

As illustrated in FIG. 3, the shaft 58 of the helix-shaft assembly 54 includes a distal shaft portion 70, a distal flange 72, an intermediate shaft portion 74, a throat engagement structure 76, a proximal shaft portion 78, and proximal flange 80. The distal flange 72 includes a distal face 73 and a proximal face 75 opposite the distal face. Both of these faces 73, 75 extend radially perpendicularly outward from their respective shaft portions 70, 74. The diameter of the distal shaft portion 70 is larger than the intermediate shaft portion 74, which is larger than the diameter of the proximal shaft portion 78. The distal flange 72 separates the distal and intermediate shaft portions. A proximal portion of the anchor 26 helically extends around the distal shaft portion 70 such that the anchor 26 is coaxial with the distal shaft portion. The proximal end of the anchor 26 abuts against the distal face 73 of the distal flange 72. The anchor 26 distally extends from distal shaft portion 70.

The helically wound conductor 29, which extends from and is electrically connected to the pin contact 24 of the connector assembly 18, helically extends about the proximal shaft portion 78. The conductor 29 abuts against the proximal face of the proximal flange 80, which is employed as a weld flange for welding the conductor 29 to the shaft 58. The conductor 29 proximally extends from the proximal shaft portion 78.

The shaft 58 may be formed of an electrically conductive material and serve as an electrical pathway leading between the helical conductor 29 and the helical anchor 26 such that the anchor 26 can serve as an electrode. Methods of establishing an electrical connection between the conductor 29 and the shaft 58 and the anchor and the shaft include, but are not limited to, welding, crimping, etc. While the embodiment depicted herein is discussed in the context of the anchor 26 also serving in an electrode capacity, in other embodiments, the anchor 26 will not have any electrode capacity and will simply serve as an anchoring mechanism.

As shown in FIG. 5, which is an enlarged view of the throat engagement structure 76 illustrated in FIG. 3, the throat engagement structure 76 includes a distal face 82, a proximal face 84 opposite the distal face 82, and a proximally tapering circumferential surface 86. The distal face 82 radially extends perpendicularly outward from the intermediate shaft portion 74. The proximal face 84 radially extends perpendicularly outward from the proximal shaft portion 78 and is opposed to and offset from a distal face of the proximal flange 80. The proximally tapering circumferential surface 86 intersects an outer edge of the proximal face 84, which is the location of the smallest diameter of the proximally tapering circumferential surface 86.

As can be understood from FIGS. 2A and 2B, which illustrate the throat engagement structure 76 engaged in a male-female sliding interlocking interference fit arrangement 90 with the throat 60, and also referring to FIGS. 3-5, the proximal face 84 of the throat engagement structure 76 has a diameter noticeably smaller than the diameter of the throat passage 68. Also, the throat engagement structure 76 has a tapered circumferential surface 86 that increases in the distal direction, thereby creating a wedge-like arrangement leading to the distal face 82 of the throat engagement structure 76. The diameter of the radially extending distal face 82 of the throat engagement structure 76 is essentially equal to the diameter of the proximal passage 62 of the header 52 and exceeds the diameter of the throat passage 68.

During assembly of the helix-shaft assembly 54 into the header 52, the helical conductor 29, and the proximal shaft portion 78 from which the conductor 29 proximally extends, are inserted as a single unit through the throat passage 68 when the lead 10 is being assembled. Once the conductor 29 has led the way through the throat passage 68, the proximal shaft portion 78 is increasingly passed proximally through the throat passage 68 until eventually the proximal face 84 of the throat engagement structure 76 enters the throat passage 68. As the throat engagement structure 76 increasingly passes proximally through the throat passage 68, the wedging action of wedge-like arrangement of the structure 76 radially forces outward the cantilevered throat structure 65 until the instant the distal face 82 of the throat engagement structure 76 clears the proximal face 67 of the lip 69 of the throat 64. At such an instant, the cantilevered throat structure 65 biases radially inward to cause the proximal face 67 of the throat lip 69 to be distally located relative to, and abutting against in opposed fashion, the distal face 82 of the throat engagement structure 76, as is the case in FIG. 2A. Thus, distal displacement of the helix-shaft assembly 54 within the header 52 once the two are coupled together via the described interference fit is limited to that depicted in FIG. 2A by the abutting of the opposed faces 82, 67.

As can be understood from a comparison of FIGS. 2A and 2B, although the described interference fit keeps the helix-shaft assembly 54 locked within the confines of the header 52 once they are assembled together, the interference fit does allow for limited sliding longitudinal displacement of the assembly 54 within the header 52. Distal sliding displacement of the assembly 54 within the header 52 is limited by the abutting of the opposed faces 82, 67, as indicated in FIG. 2A. Similarly, proximal sliding displacement of the assembly 54 within the header 52 is limited by the abutting of the opposed faces 75, 66, as illustrated in FIG. 2B. Depending on the deployment arrangement and method employed in causing the helix-shaft assembly 54 to transition from the non-deployed configuration of FIG. 2B to the deployed configuration of FIG. 2A, the helix-shaft assembly 54 may additionally rotate about its longitudinal axis within the confines of the header 52.

In summary, in one embodiment, the helix-shaft assembly is designed to fit into the header and snap-lock into the internal geometry of the header. The snap-lock features in the header keep the shaft and the helix extending therefrom within the header when extending the shaft and helix during lead implant. The sloped features on the shaft are designed to deflect the snap-lock features of the header throat when inserted into the header. Once the shaft features pass the header throat features, the snap-lock securing of the shaft within the header occurs such that the shaft cannot be removed from the header.

2. Engagement of Header and Marker-Tip Assembly

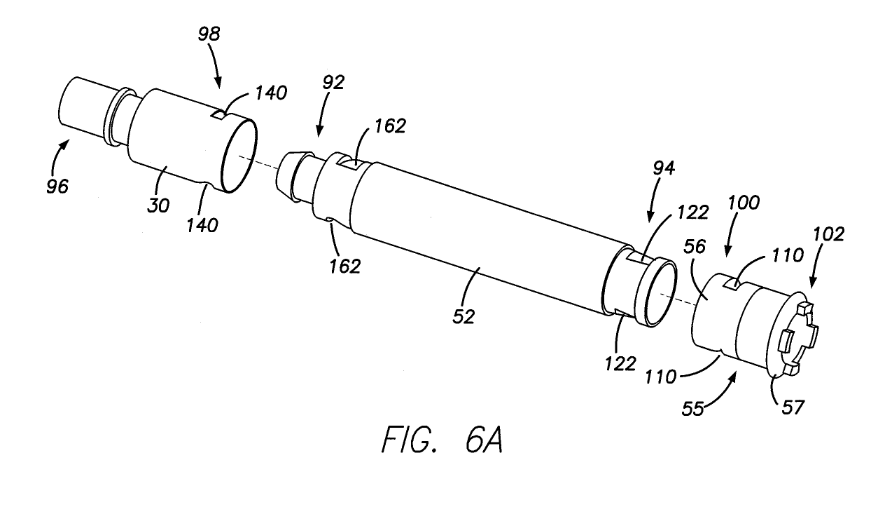

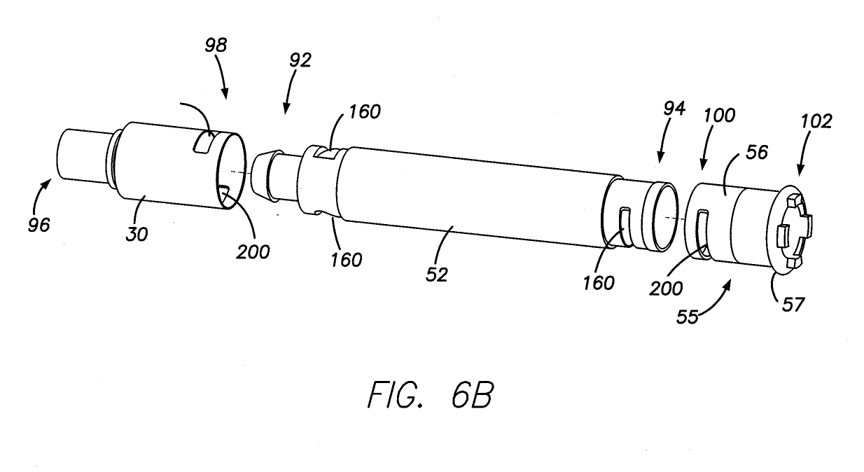

FIG. 6A is an exploded isometric view of the of the ring electrode 30, header 52 and the marker tip assembly 55, wherein the header 52 has female snap-lock features and the ring electrode 20 and the marker tip assembly 55 have complementary male snap-lock features. FIG. 6B is a view similar to that of FIG. 6A, except of another embodiment wherein the header 52 has male snap-lock features and the ring electrode 20 and the marker tip assembly 55 have complementary female snap-lock features.

As shown in FIGS. 6A and 6B, the header 52 includes a proximal end 92 and a distal end 94, the ring electrode 30 includes a proximal end 96 and a distal end 98, and the marker tip assembly 55 includes a proximal end 100 and a distal end 102. As can be understood from FIGS. 2A, 2B, 6A, and 6B, the proximal end 92 of the header 52 is a male component, and the distal end 98 of the ring electrode 30 is a female component in which the header proximal end 92 is received. The snap-lock features supported on these ends 92, 98 interlock to maintain the male proximal end 92 of the header 52 within the confines of the female distal end 98 of the ring electrode 30.

The distal end 94 of the header 52 is a male component, and the proximal end 100 of the marker tip assembly 55 is a female component in which the header distal end 94 is received. The snap-lock features supported on these ends 94, 100 interlock to maintain the male distal end 94 of the header 52 within the confines of the female proximal end 100 of the marker tip assembly 55.

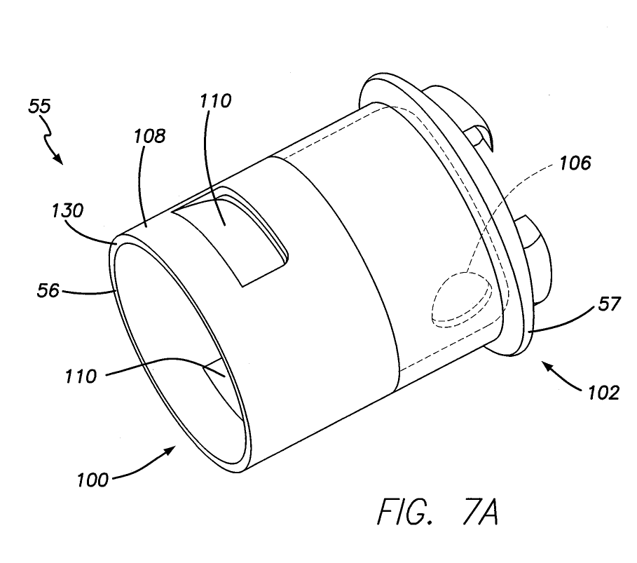

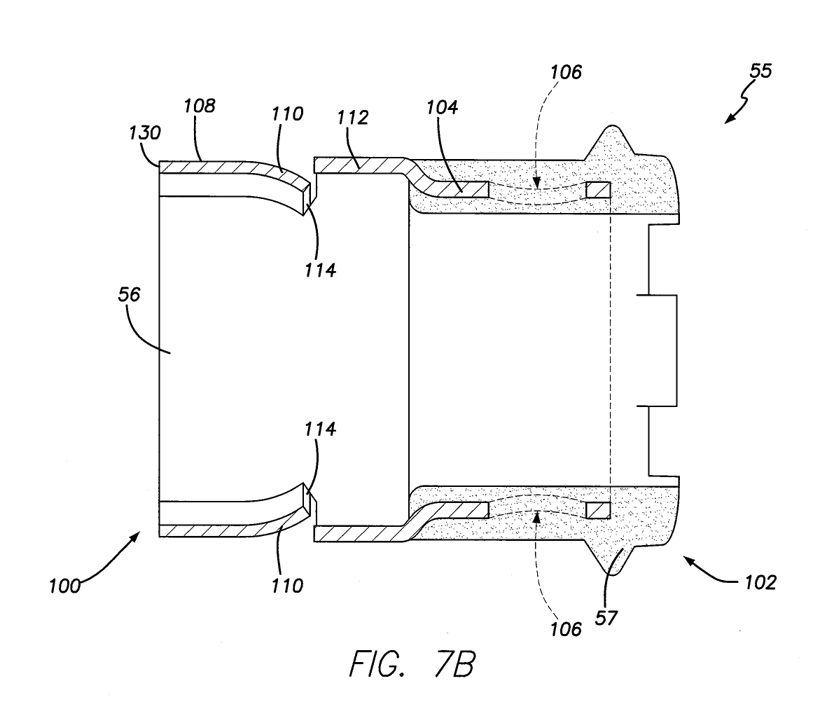

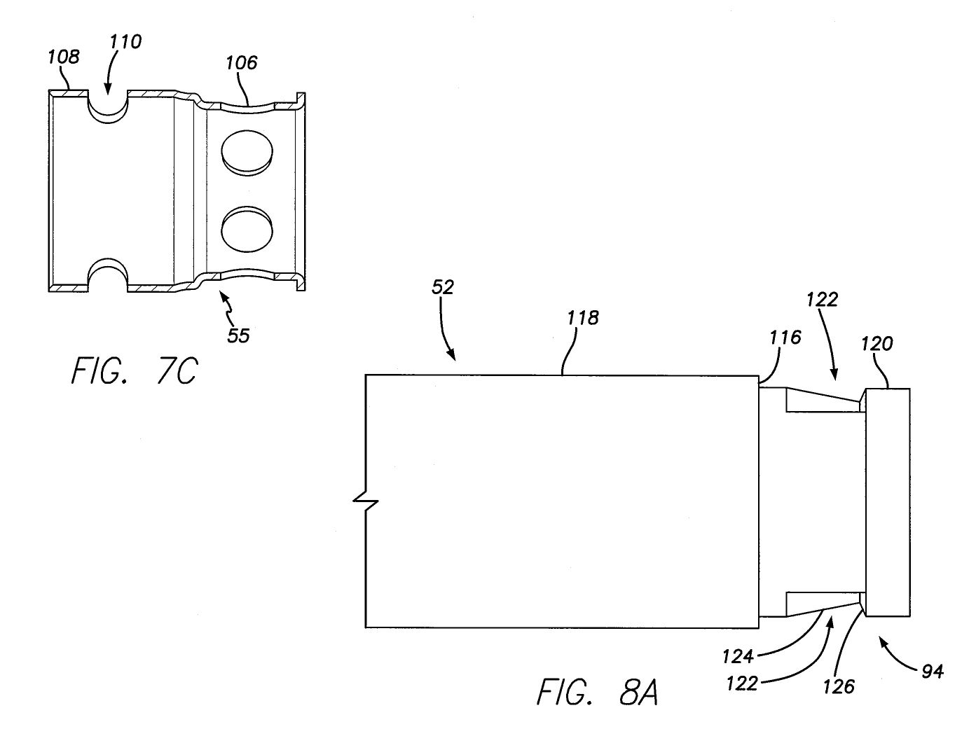

As shown in FIGS. 7A and 7B, which are, respectively, isometric and longitudinal cross sectional views of the marker tip assembly 55, the assembly 55 includes a marker ring 56 and a soft atraumatic tip 57 extending distally therefrom. The marker ring 56 is formed of a biocompatible radiopaque material such as, for example, platinum, gold, stainless steel, or etc. The marker ring 56 may be a stamped-progressive formed part. The soft atraumatic tip 57 is formed of a biocompatible polymer such as, for example, silicone rubber, polyurethane, SPC, or etc. The atraumatic tip 57 may be overmolded over a distal cylindrical region 104 of the marker ring 56, the soft polymer material forming the atraumatic tip 57 even extending through holes 106 defined in the circumference of the distal region 104.

The marker ring 56 includes a proximal cylindrical region 108 in which male snap-lock features 110 are defined. While the snap-lock features 110 of the marker ring 56 of FIGS. 6A, 7A and 7B are configured to be male snap-lock features 110, as can be understood from FIG. 6B the snap-lock features of the marker ring 56 can be configured to be female snap-lock features.

As indicated in FIGS. 7A and 7B, the male snap-lock features 110 may be in the form of cantilevered tabs or projections 110 that are defined out of the cylindrical wall 112 forming the proximal cylindrical region 108, each cantilevered projection 110 having a free end 114 that extends distally and biases radially inward. The cantilevered projections 110 may be laser cut, stamped or otherwise defined out of the cylindrical wall 112.

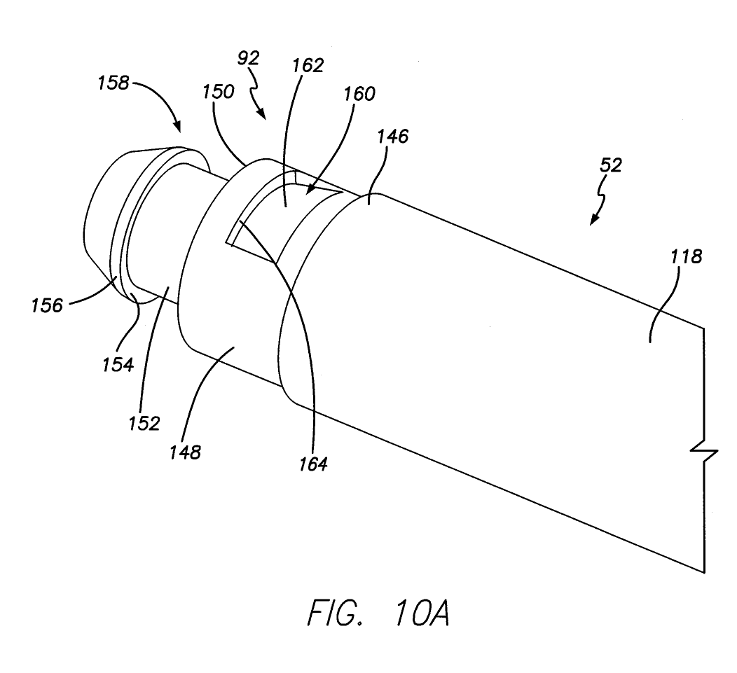

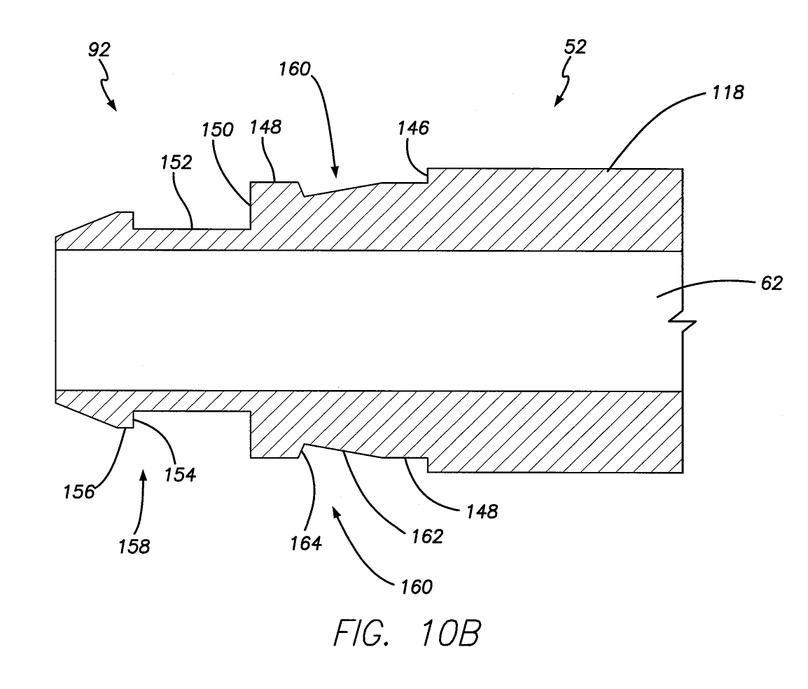

As mentioned above with respect to FIG. 6B, the snap-lock features 110 of marker ring 55 can be a female snap-lock feature 110 in the form of a cutout 110, as depicted in FIG. 7C, which is a longitudinal cross section of the marker ring 55 with an alternative embodiment of its snap-lock feature 110. Such a female snap-lock feature 110 as depicted in FIG. 7C could be employed with a male snap-lock feature similar to those depicted in FIGS. 6B and 10C.

As shown in FIGS. 8A and 8B, which are, respectively, side elevation and longitudinal cross sectional views of the distal region of the header 52 distally terminating in the header distal end 94, the distal region includes a stepped arrangement 116 that transitions from the diameter of a middle cylindrical portion 118 to the smaller diameter of a distal cylindrical portion 120 forming the header distal end 94. A distal portion of the above-discussed distal cylindrical passage 60 of the header 52 can be seen in FIG. 8B.

Female snap-lock features 122 are defined in the distal cylindrical portion 120. While the snap-lock features 122 of the header distal end 94 of FIGS. 6A, 7A and 7B are configured to be female snap-lock features 122, as can be understood from FIG. 6B the snap-lock features of the header distal end 94 can be configured to be male snap-lock features.

As indicated in FIGS. 8A and 8B, the female snap-lock features 122 may be in the form of radially inwardly sloping features 122 that are defined in the outer surface of the distal cylindrical portion 120. Each sloping feature 122 has a slope surface 124 that slopes increasingly radially inwardly extending proximal to distal, thereby transitioning from the outer surface of the distal cylindrical portion 120 to intersect a radially inward boundary of a radially outwardly extending face 126 that faces proximally. The header 52 may be formed from a polymer such as, for example, PEEK. Accordingly, the snap-lock features 122 may be molded as part of the formation of the header 52 from PEEK. Alternatively, the snap-lock features may be milled or otherwise machined into the header 52.

As can be understood from FIGS. 2A and 2B, which illustrate the interlocking of the snap-lock features 110, 122 of the marker ring 56 and the distal header end 94, the proximal end 100 of the marker tip assembly 55 receives the distal header end 94 in a male-female arrangement. The inner circumferential surface of the marker ring 56 abuts in generally continuous circumferential contact with the outer circumferential surface of the distal cylindrical portion 120 forming the distal header end 94, and the male snap-lock features 110 are received in an interlocking arrangement with the female snap-lock features 122.

During assembly of the proximal end 100 of the marker tip assembly 55 onto the distal end 94 of the header 52, the header distal end 94 is inserted into the proximal end 100 of the marker tip assembly 55. As the distal end 94 is increasingly inserted into the proximal end 100, the free ends 114 of the respective male snap-lock features 110 slide along the outer cylindrical surface of the distal cylindrical portion 120 of the header distal end 94 until reaching the proximal faces 126 of the female snap-lock feature 122, at which time the free ends 114 drop into the recesses of the respective female snap-lock features 122 to abut against the associated proximal faces 126, thereby preventing the marker tip assembly 55 and the header distal end 94 from longitudinally displacing away from each other. Generally simultaneous abutting contact of a proximal edge 130 of the marker ring 56 against the step arrangement 116 of the header 52 prevents the header distal end 94 from being received deeper into the marker tip assembly 55. As a result of this abutting contact and the snap-lock interfacing of the features 110, 122, the maker tip assembly 55 is interlocked onto the header distal end 94.

3. Engagement of Header and Ring Electrode

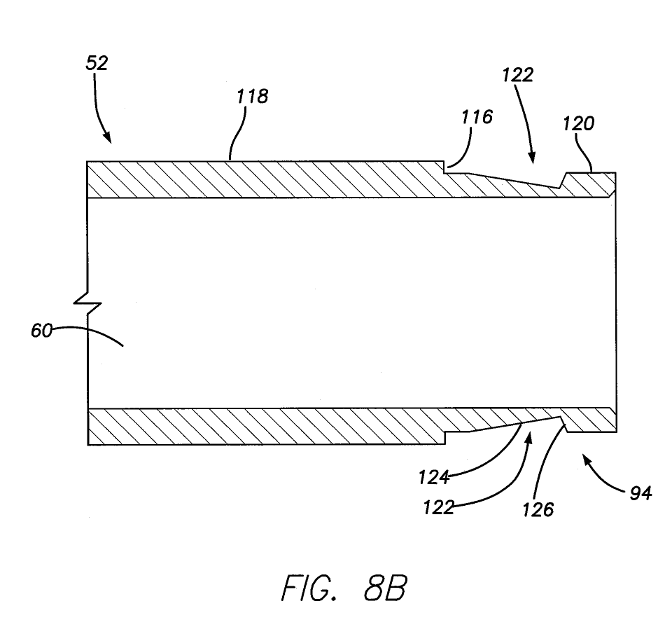

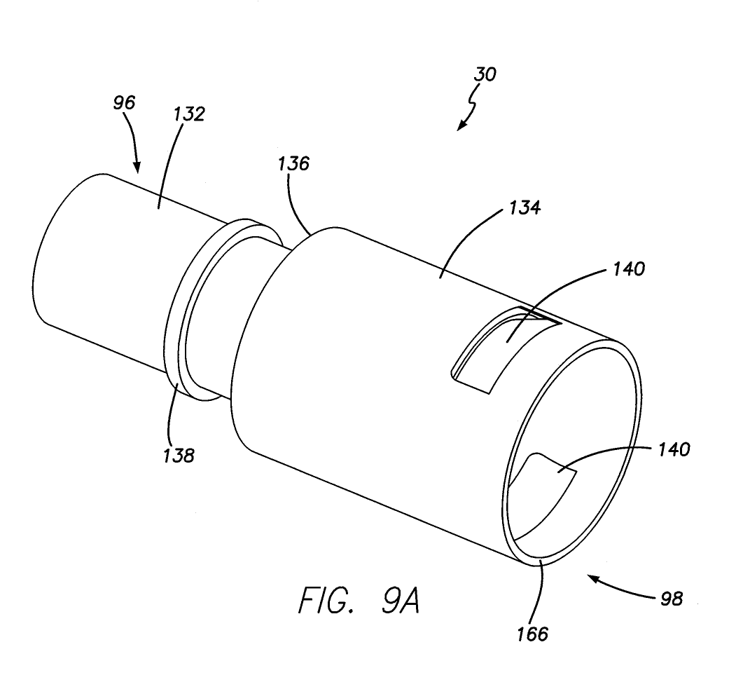

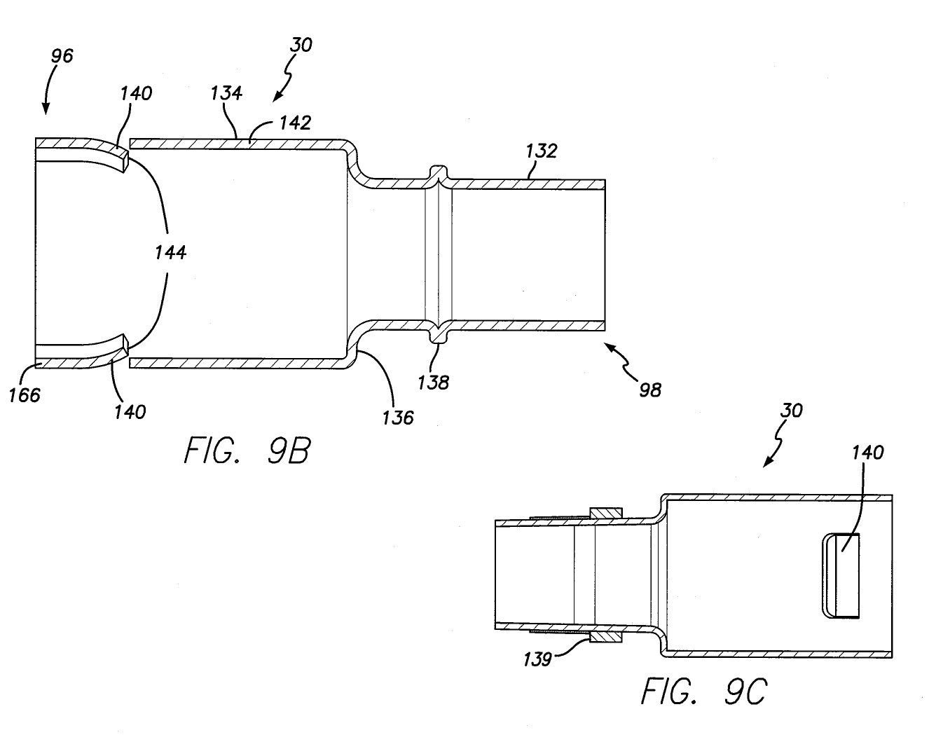

As shown in FIGS. 9A and 9B, which are, respectively, isometric and longitudinal cross sectional views of the ring electrode 30, the ring electrode 30 includes a proximal end 96 and a distal end 98. The ring electrode 30 also includes a proximal cylindrical region 132, a distal cylindrical region 134 that is larger in diameter than the proximal cylindrical region 132, and a stepped transition 136 between the two cylindrical regions 132, 134. The ring electrode 30 is formed of a biocompatible electrically conductive material such as, for example, platinum, platinum-iridium alloy, stainless steel, or etc. The ring electrode 30 may be a stamped-progressive formed part.

The proximal cylindrical region 132 includes a radial flange 138 located about two-thirds of the length of the proximal cylindrical region 132 from the most proximal edge of the proximal cylindrical region 132. As can be understood from FIGS. 2A and 2B, the outer helical conductor 32 is wound about the outer circumferential surface of the proximal cylindrical region 132 between the most proximal edge of the region 132 and the proximal face of the radial flange 138. The flange 138 may serve as a weld collar 138 for welded attachment to the helical conductor 32. The outer insulation layer 38 extends over the outer helical conductor 32, the outer edge of the flange 138, and the rest of the proximal cylindrical region 132 to abut against the stepped transition 138.

As can be understood from FIGS. 9A and 9B, in one embodiment the flange 138 is formed into the ring electrode 30 via the stamped-progressive formation of the ring electrode. In another embodiment, the flange 138 is not formed, but a press-fit weld collar 139 is employed, as illustrated in FIG. 9C.

Referring again FIGS. 9A and 9B, male snap-lock features 140 are defined in the distal cylindrical region 134. While the snap-lock features 140 of the ring electrode 30 of FIGS. 6A, 9A and 9B are configured to be male snap-lock features 140, as can be understood from FIG. 6B the snap-lock features of the ring electrode 30 can be configured to be female snap-lock features.

As indicated in FIGS. 9A and 9B, the male snap-lock features 140 may be in the form of cantilevered tabs or projections 140 that are defined out of the cylindrical wall 142 forming the distal cylindrical region 134, each cantilevered projection 142 having a free end 144 that extends proximally and biases radially inward. The cantilevered projections 140 may be laser cut, stamped or otherwise defined out of the cylindrical wall 142.

As shown in FIGS. 10A and 10B, which are, respectively, isometric and longitudinal cross sectional views of the proximal region of the header 52 proximally terminating in the header proximal end 92, the proximal region includes a first stepped arrangement 146 that transitions from the diameter of the middle cylindrical portion 118 to the smaller diameter of a first proximal cylindrical portion 148 forming a portion of the header proximal end 92. The proximal region also includes a second stepped arrangement 150 that transitions from the diameter of the first proximal cylindrical portion 148 to the smaller diameter of a second proximal cylindrical portion 152 forming another portion of the header proximal end 92. The proximal region still further includes a third stepped arrangement 154 that transitions from the diameter of the second proximal cylindrical portion 152 to the larger diameter of a barbed portion 156 that tapers proximally and forms another portion of the header proximal end 92. The barbed portion 156 and the second proximal cylindrical portion 152 combine to define a barbed connector portion 158. As can be understood from FIGS. 2A and 2B, the barbed connector portion 158 is received in a distal end of the inner insulation layer 39. The inner insulation layer 39 may be heat-shrunk about barbed connector portion 158 or otherwise secured to the barbed connector portion 158.

A proximal portion of the above-discussed proximal cylindrical passage 62 of the header 52 can be seen in FIG. 10B. As illustrated in FIGS. 10A and 10B, female snap-lock features 160 are defined in the first proximal cylindrical portion 148. While the snap-lock features 160 of the header proximal end 92 of FIGS. 6A, 10A and 10B are configured to be female snap-lock features 160, as can be understood from FIG. 6B the snap-lock features of the header proximal end 92 can be configured to be male snap-lock features.

As indicated in FIGS. 10A and 10B, the female snap-lock features 160 may be in the form of radially inwardly sloping features 160 that are defined in the outer surface of the first proximal cylindrical portion 148. Each sloping feature 160 has a slope surface 162 that slopes increasingly radially inwardly extending distal to proximal, thereby transitioning from the outer surface of the first proximal cylindrical portion 148 to intersect a radially inward boundary of a radially outwardly extending face 164 that faces distally. The header 52 may be formed from a polymer such as, for example, PEEK. Accordingly, the snap-lock features 160 may be molded as part of the formation of the header 52 from PEEK. Alternatively, the snap-lock features may be milled or otherwise machined into the header 52.

As can be understood from FIGS. 2A and 2B, which illustrate the interlocking of the snap-lock features 140, 160 of the ring electrode 30 and the proximal header end 92, the distal end 98 of the ring electrode 30 receives the proximal header end 92 in a male-female arrangement. The inner circumferential surface of the ring electrode 30 abuts in generally continuous circumferential contact with the outer circumferential surface of the first proximal cylindrical portion 148 forming part of the proximal header end 92, and the male snap-lock features 140 are received in an interlocking arrangement with the female snap-lock features 160.

During assembly of the distal end 98 of the ring electrode 30 onto the proximal end 92 of the header 52, the header proximal end 92 is inserted into the distal end 98 of the ring electrode 30. As the proximal end 92 is increasingly inserted into the distal end 98, the free ends 144 of the respective male snap-lock features 140 slide along the outer cylindrical surface of the first proximal cylindrical portion 148 of the header proximal end 92 until reaching the distal faces 164 of the female snap-lock feature 160, at which time the free ends 144 drop into the recesses of the respective female snap-lock features 160 to abut against the associated distal faces 164, thereby preventing the ring electrode 30 and the header proximal end 92 from longitudinally displacing away from each other. Generally simultaneous abutting contact of a distal edge 166 of the ring electrode 30 against the step arrangement 146 of the header 52 prevents the header proximal end 92 from being received deeper into the ring electrode 30. As a result of this abutting contact and the snap-lock interfacing of the features 140, 160, the ring electrode 30 is interlocked onto the header proximal end 92.

As mentioned above with respect to FIG. 6B, the snap-lock features 160 of the header proximal end 92 can be a male snap-lock feature 160, as depicted in FIG. 10C, which is a side view of the header proximal end 52 with an alternative embodiment of its snap-lock feature 160. Such a male snap-lock feature 160 as depicted in FIG. 10C could be employed with the male snap-lock feature 140 of FIGS. 9A and 9B or with a female snap-lock feature such as, for example, the cutout 110 in FIG. 7C or as depicted in the ring electrode 30 of FIG. 6B.

Regardless of whether the snap-lock features are female or male such that the snap-lock features utilize cut-outs and tabs or some other snap-lock configuration, snap-lock mating and securing arrangements may be employed to connect the header to the ring electrode, the header to the marker ring, and the helix-shaft assembly to the header. Further, the snap-lock features may be applied to any lead components that need to be attached to each other. The header is molded out of PEEK material with the snap-lock features incorporated.

In one embodiment, as can be understood from FIGS. 7C, 9A, 9B and 10C, the snap-lock features on the header are male tabs 160 that are received in and interface with female cut-outs 110 in the maker ring and punched tabs 140 in the ring electrode. These snap-lock features employ vertical faces and sloped surfaces that allow the components to be slipped together and then engage in a locked relationship. For example, in one embodiment, the sloped surface of the snap-lock feature that interacts with the locking tab on the ring electrode slightly deflects the tab to allow the ring electrode to be fit over the header end.

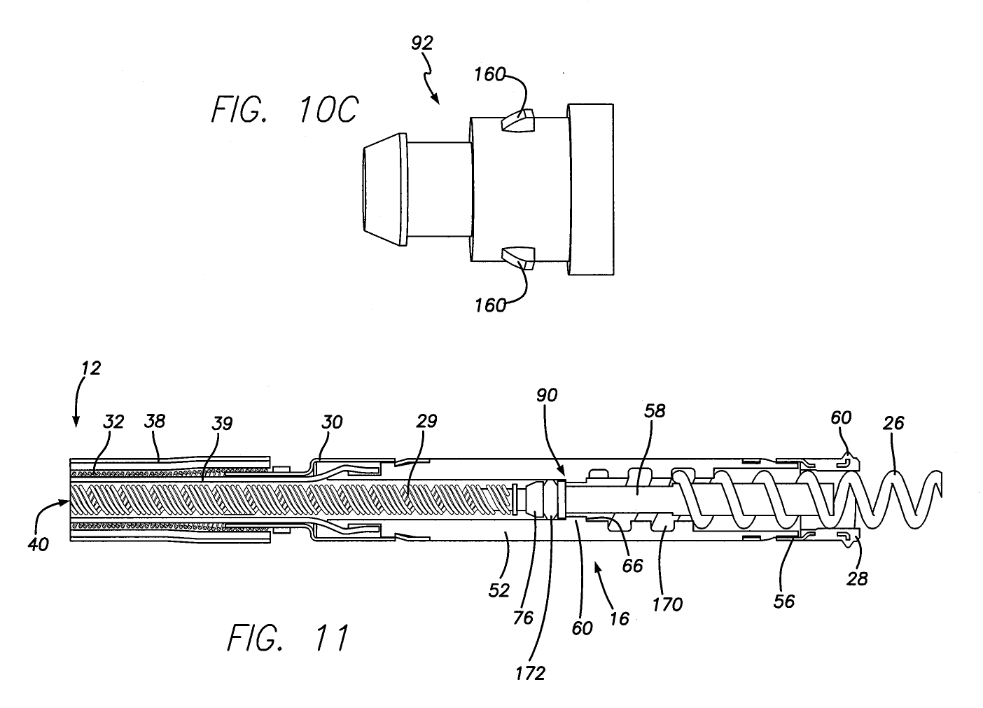

As can be understood from FIGS. 2A and 2B, in one embodiment, the lead distal tip assembly employs a header post design for deploying the helical anchor 26. However, as depicted in FIG. 11, which is the same view as FIG. 2A, except of an alternative embodiment, the lead distal tip assembly employs a post-less design. The interior of the header 52 has helical threads 170 with which the coils of the helical anchor 26 interface in threaded engagement during the transition of the anchor 26 between non-deployed and deployed states. As can be understood from a comparison of FIGS. 2A, 2B and 11, the shaft 58 and header 52 of the embodiment of FIG. 11 have similar features that allows for the shaft 58 to be press or snap-lock into the header 52 as described above for the creation of a male-female sliding interlocking interference fit arrangement 90 with the throat 60.

A blood seal 172 is as part of the throat engagement structure 76 of the shaft 58 and may be formed of silicone rubber, polyurethane, SPC, or other suitable polymer. The blood seal 172 prevents the ingress of blood into the lead inner coil 29 during implant and chronic use. The may be in an O-ring design with a peaked ridge that interacts with the inner surface of the header 52. The blood seal 172 is configured such that it will have minimal interference with the helix extension and retraction functionality.

Some versions of the above-described embodiments of the snap-lock designs may be configured so as to prevent the components from being separated after they are pressed together. Other versions of the above-described embodiments of the snap-lock designs may be configured so as to allow the components to be separated after they are pressed together. For example, the snap-lock designs may include deflectable members or aspects, which when pressed or pressed together, cause the snap-lock features to deflect so as to allow the components to separate for repositioning.

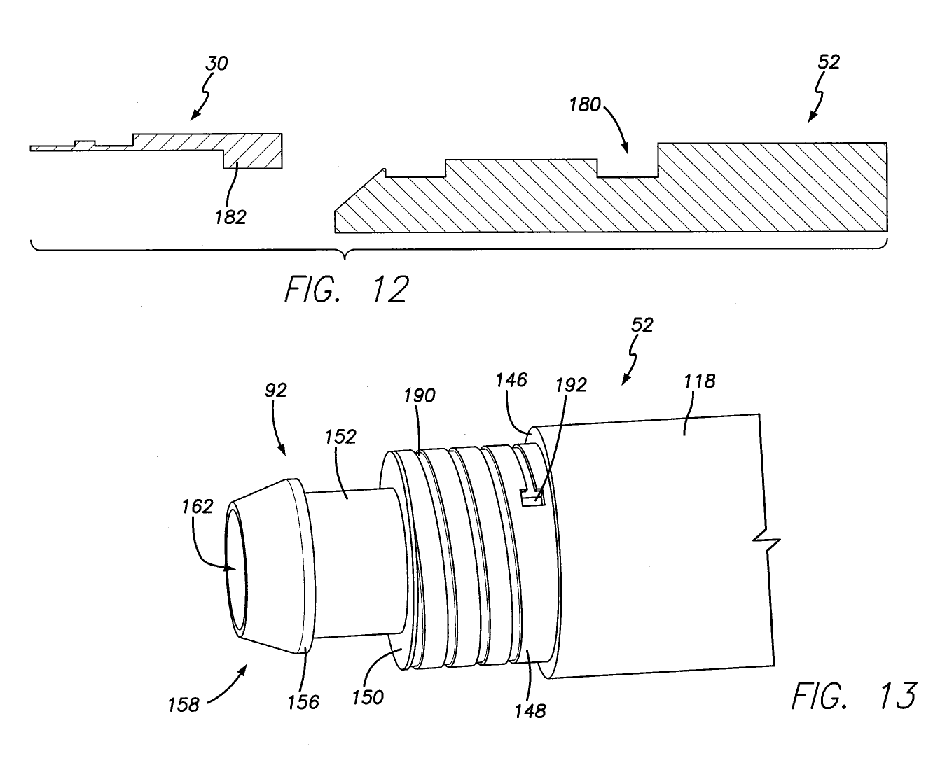

As can be understood from FIG. 12, which is a cross section of a snap-lock arrangement, the snap-lock arrangement includes a divot or depression 180 on the header 52 that interacts with a locking tab 182 on the ring electrode 30 instead of a raised feature. The locking tab 182 slides across the surface of the mating component 52 and then falls into a locking hole 180 that prevents the components pulling apart or rotating relative to each other.

As can be understood from FIG. 13, which is an isometric view of another embodiment of the header proximal end 92, the end 92 includes threading 190 defined in the outer circumferential surface of the first proximal cylindrical region 148. A locking feature 192 is defined at the end of the threading 190. The complementary ring electrode 30 includes complementary threads or other features that will be threadably received in the threading 190 as the ring electrode is threaded over the header proximal end 92. As the ring electrode is fully threaded onto the header proximal end 92, a male snap-lock component is received in the locking feature 192, thereby preventing reverse threading of the ring electrode from the header proximal end 192, thereby preventing separation of the ring electrode and the header proximal end.

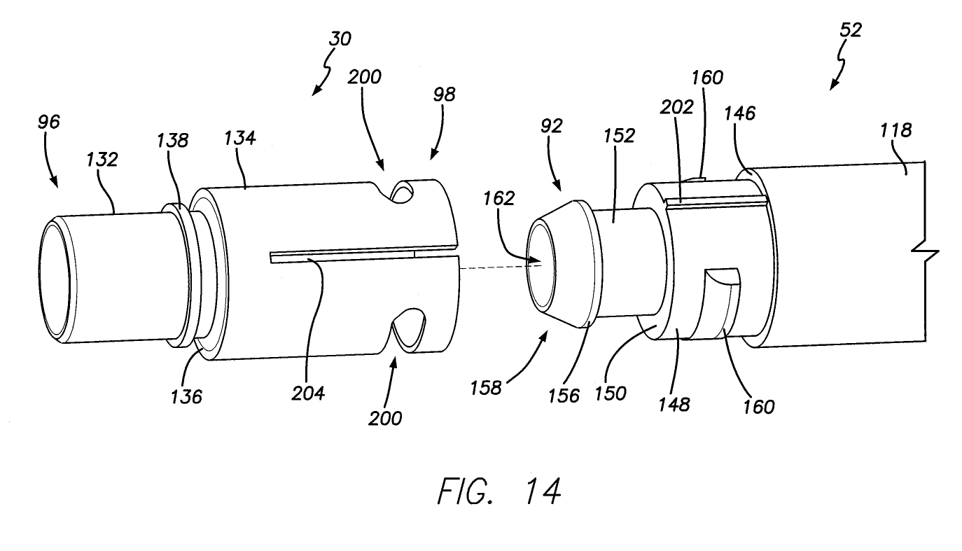

As indicated in FIG. 14, which is an exploded isometric view of additional embodiments of the electrode ring 30 and header proximal end 92, the snap-lock arrangement formed by the tabs 160 and the holes or cutouts 200 are supplemented by one or more male guide features 202 and one or more complementary female guide features 204 that are radially offset from the tables 160 and cutouts 200 by approximately 90°. In other words, in one embodiment, the ring electrode 30 has at least one guide channel 204 and at least one the snap-lock feature (e.g., cutout 200), and the header proximal end 92 has at least one guide ridge 202 and at least one the snap-lock feature (e.g., tab 160). Each ridge 202 is received in a corresponding channel 204 as each tab 160 is received in a corresponding cutout 200. The interfacing of the channels 204 with the corresponding ridges 202 can help ensure that the snap-lock features 160, 200 fully interact and secure the components 30, 52 together. The snap-lock features 160, 200 prevent the two components 30, 52 from pulling apart from each other, and the guide features 202, 204 prevent the two components from rotating relative to each other.

In one embodiment, the ring electrode 30 will be stamped-progressive formed with tabs 140 punched out that will snap-lock with complementary features 162 on the header 52, as can be understood from FIG. 6A. In another embodiment, the ring electrode 30 will be stamped-progressive formed with cutouts 200 punched out that will snap-lock with complementary male features 160 on the header 52, as can be understood from FIG. 6B. As noted above, the ring electrode can have a formed weld collar (see FIG. 9B) or a press-fit weld collar (see FIG. 9C). The marker ring 56 can also be stamp-progressive formed with tabs 110 (see FIG. 6A) or cutouts 200 (see FIG. 6B) to respectively mate with features 122, 160 on the header distal end 94. The soft atraumatic tip 57 can be overmolded onto the marker ring 56.

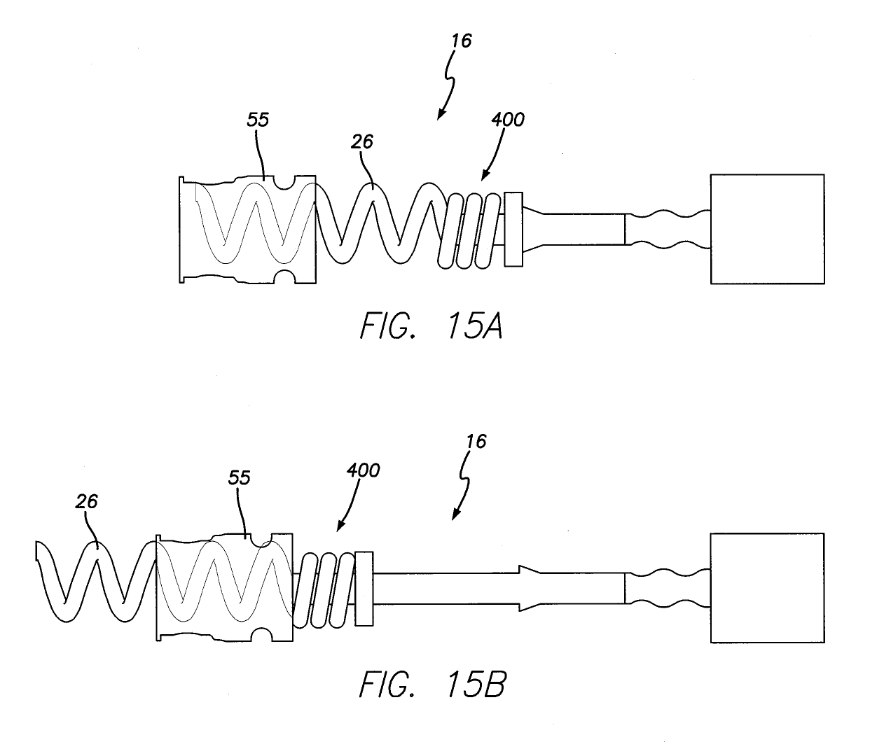

FIG. 15A is a side elevation view of the distal region 16 of the lead 10 via fluoroscopic visualization, wherein the helical anchor 26 is shown in the non-deployed or fully retracted state. FIG. 15B is the same view as FIG. 15A, except the helical anchor 26 is shown in the fully-deployed state or fully extended state. These figures illustrate the range of complete extension of the helical anchor 26 for one embodiment of the lead. The design enhancement provided by the helix-shaft assembly and the marker ring assembly 55 allows for the determination of complete helix extension under fluoroscopy, as can be understood from FIGS. 15A and 15B. The helix-shaft assembly incorporates a section of tightly pitched coils 400 of the helical anchor 26 that can be used as a visual marker. As indicated in FIG. 15B, the helical anchor 26 is fully extended when the distal edge of the tightly pitched coils 400 are next to the proximal edge of the marker ring assembly 55. Conversely, when there is a gap between the proximal edge of the marker ring assembly 55 and the distal edge of the tightly pitched coils 400 and the distal tip of the helical anchor 26 is proximal the distal edge of the marker ring assembly 55, then the helical anchor 26 is retracted within the header, as can be understood from FIG. 15A.

In general, while the invention has been described with reference to particular embodiments, modifications can be made thereto without departing from the spirit and scope of the invention. Note also that the term “including” as used herein is intended to be inclusive, i.e. “including but not limited to.”

What is claimed is:

1. An implantable therapy lead comprising:
an integrated distal tip assembly including a header;
wherein at least one of a helix-shaft assembly, a ring electrode or a marker ring is directly coupled to the header via a snap-lock coupling arrangement.

2. The lead of claim 1, wherein the helix-shaft assembly, in addition to being directly coupled to the header via the snap-lock coupling arrangement, is also longitudinally displaceable relative to the header.

3. The lead of claim 1, wherein the snap-lock coupling arrangement between the header and the helix-shaft assembly includes a tapered male member on the helix-shaft assembly and a female throat in the header, the female throat having received, and snap-locked with, the tapered male member during an assembly process.

4. The lead of claim 3, wherein the helix-shaft assembly includes a flexible O-ring supported on the helix-shaft assembly adjacent the tapered male member and in a sliding sealed engagement with the header proximal the female throat.

5. The lead of claim 3, wherein the female throat includes a cantilevered structure that was forced radially outward by the passage of the tapered male member through the female throat during the assembly process, the cantilevered structure having biased radially inward to snap-lock with the tapered male member after the tapered male member cleared the cantilevered structure.

6. The lead of claim 5, wherein the cantilevered structure distally projects, and the tapered male member tapers in a proximal direction.

7. The lead of claim 1, wherein the snap-lock coupling arrangement between the header and the ring electrode comprises: a male end of the header received in a female end of the ring electrode; and a radially inwardly biased tab defined in the ring electrode that is received in, and snap-locked with, an opening or recess defined in the male end of the header.

8. The lead of claim 7, wherein the radially inwardly biased tab has a cantilevered configuration that includes a proximally facing free end that is received in, and snap-locks with, the opening or recess defined in the male end of the header.

9. The lead of claim 1, wherein the snap-lock coupling arrangement between the header and the ring electrode comprises: a male end of the header received in a female end of the ring electrode; and a male tab defined in the male end of the header that is received in, and snap-locked with, an opening or recess defined in the ring electrode.

10. The lead of claim 9, wherein the male tab comprises a sloped proximal surface.

11. The lead of claim 1, wherein the snap-lock coupling arrangement between the header and the marker ring comprises: a male end of the header received in a female end of the marker ring; and a radially inwardly biased tab defined in the marker ring that is received in, and snap-locked with, an opening or recess defined in the male end of the header.

12. The lead of claim 11, wherein the radially inwardly biased tab has a cantilevered configuration that comprises a distally facing free end that is received in, and snap-locks with, the opening or recess defined in the male end of the header.

13. The lead of claim 1, wherein the snap-lock coupling arrangement between the header and the marker ring comprises: a male end of the header received in a female end of the marker ring; and a male tab defined in the male end of the header that is received in, and snap-locked with, an opening or recess defined in the marker ring.

14. The lead of claim 13, wherein the male tab comprises a sloped distal surface.

15. The lead of claim 1, wherein an atraumatic tip is overmolded on the marker ring.

16. The lead of claim 1, wherein the snap-lock coupling arrangement between the header and the ring electrode comprises: a male end of the header received in a female end of the ring electrode; and a radially inwardly biased tab defined in the ring electrode that is received in, and snap-locked with, an opening or recess defined in the male end of the header, the opening intersecting a thread feature defined in the male end of the header.

17. The lead of claim 1, wherein the snap-lock coupling arrangement between the header and the marker ring comprises: a male end of the header received in a female end of the marker ring; and a radially inwardly biased tab defined in the marker ring that is received in, and snap-locked with, an opening or recess defined in the male end of the header, the opening intersecting a thread feature defined in the male end of the header.

18. The lead of claim 1, wherein the snap-lock coupling arrangement between the header and the marker ring comprises: a male end of the header received in a female end of the ring electrode; and a first longitudinally extending structural feature on the marker ring that mates with, and slides along, a second longitudinally extending structural feature of the male end of the header.

19. The lead of claim 18, wherein the first longitudinally extending structural feature comprises a slot, and the second longitudinally extending structural feature comprises a raised ridge.

20. The lead of claim 1, wherein the snap-lock coupling arrangement between the header and the ring electrode comprises: a male end of the header received in a female end of the ring electrode; and a first longitudinally extending structural feature on the ring electrode that mates with, and slides along, a second longitudinally extending structural feature of the male end of the header.

21. The lead of claim 18, wherein the first longitudinally extending structural feature comprises a slot, and the second longitudinally extending structural feature comprises a raised ridge.

22. A method of assembling an integrated distal tip assembly of an implantable therapy lead, the method comprising directly coupling at least one of a helix-shaft assembly, a ring electrode or a marker ring to a header via a snap-lock coupling arrangement.

23. The method of claim 22, further comprising a helical conductor proximally extending from a proximal region of the helix-shaft assembly, and the helix-shaft assembly is directly coupled to the header by extending a proximal end of the helical conductor through a throat of the header followed by proximally extending the helix-shaft assembly through the throat until a tapered male member of the helix-shaft passes through the throat and engages with the throat in the snap-lock coupling arrangement.

24. The method of claim 22, further comprising inserting a distal male end of the header into a proximal female end of a marker ring, the snap-lock coupling arrangement between the header and the marker ring occurring as at least one of: a male tab of the marker ring is received in a recess or opening of the header; or a male tab of the header is received in a recess or opening of the marker ring.

25. The method of claim 22, further comprising inserting a proximal male end of the header into a distal female end of a ring electrode, the snap-lock coupling arrangement between the header and the ring electrode occurring as at least one of: a male tab of the ring electrode is received in a recess or opening of the header; or a male tab of the header is received in a recess or opening of the ring electrode.
